(19) United States
(12) Patent Application Publication
(10) Pub. No.: US 2026/0145009 A1
(43) Pub. Date: May 28, 2026

(54) Improvements in or Relating to Medical Device Apparatus

(71) Applicant: XStrahl Limited, Walsall (GB)

(72) Inventors: Emily Louise McNee, Armitage (GB); Shaun Melvyn Phillips, Peterborough (GB); Andrew James Cowie, Glen Parva (GB); Bruce James Renfrew, Leicester (GB)

(73) Assignee: XStrahl Limited, Walsall (GB)

(21) Appl. No.: 19/120,866

(22) PCT Filed: Oct. 10, 2023
(86) PCT No.: PCT/GB2023/052629

§ 371 Date: Apr. 14, 2025

(30) Foreign Application Priority Data
Oct. 14, 2022 (GB) 2215242.5

(51) Int. Cl. A61N 5/10

(52) U.S. Cl. CPC A61N 5/1077; A61N 2005/1092

(57) ABSTRACT

A mounting apparatus for a medical device includes: a housing support member; a medical device engagement member that is moveably engageable with the housing support member; and an adjustment device. The adjustment device moves the medical device engagement member relative to the housing support member. A medical device system, a support structure for a medical device system including the mounting apparatus and associated methods are also provided.

The present invention relates to a mounting apparatus for a medical device, a support structure for a medical device system, a medical device system, a mounting apparatus for a device, and a method of adjusting the position of a medical device.

X-ray systems for delivering X-ray radiotherapy for superficial therapy and treatment of skin conditions are known. Such X-ray systems include base units upon which X-ray head support arms are mounted. The base unit is typically provided on wheels or castors, which allows the X-ray system to be easily transported. The X-ray head support arm is also arranged to pivot and rotate relative to the base unit, such that the X-ray head can be positioned to be as close as possible to the patient. The X-ray head is often used with an applicator, which contacts the skin of the patient to ensure that a predetermined distance exists between the X-ray head and the skin. The applicator is also used to flatten the area to be exposed to the X-rays.

Typically, with the X-ray head positioned close to the patient's skin, the patient is moved closer to the X-ray head by, for example, adjusting the position of the patient on a bed or chair, or moving the bed or chair closer to the X-ray head.

While such X-ray systems are capable of delivering X-ray radiotherapy to patients, the repositioning of the patient after the X-ray head is in place is difficult and time consuming. Furthermore, positioning the patient incorrectly may lead to X-rays being delivered to an area of skin which is not to be treated with X-rays, which is potentially harmful to the patient.

The present inventors have appreciated the shortcomings in the above-described X-ray systems.

According to a first aspect of the present invention there is provided a mounting apparatus for a medical device, comprising:
a housing support member;
a medical device engagement member, the medical device engagement member being moveably engageable with the housing support member; and
an adjustment device, the adjustment device being configured to move the medical device engagement member relative to the housing support member.

The mounting apparatus may be a support apparatus. The mounting apparatus may be configured to provide support to a medical device.

The mounting apparatus may be configured to provide vertical support to a medical device. The mounting apparatus may be configured to provide vertical and/or horizontal support to a medical device.

The mounting apparatus may be engageable with a medical device. The mounting apparatus may be configured to engage with a medical device. The mounting apparatus may be configured to be engageable with a medical device.

The mounting apparatus may be configured to be engageable with a support structure of a medical device system. The mounting apparatus may be detachably engageable with a support structure of a medical device system. The mounting apparatus may be pivotably attachable to a support structure of a medical device system. The mounting apparatus may pivot in at least two axes with respect to the support structure of a medical device system.

The mounting apparatus may be attachable to an attachment member of a support arm of a medical device system. The mounting apparatus may be pivotably attachable to an attachment member of a support arm of a medical device system. The attachment member of the support arm of the medical device system may be pivotably attachable to the support arm. The mounting apparatus may pivot about an axis which is orthogonal to the axis about which the attachment member pivots with respect to the support arm of the medical device system. The attachment member of the support arm may be a yoke member.

The mounting apparatus may be an adjustable mounting apparatus. The position of the medical device engagement member may be adjustable relative to the housing support member. The medical device engagement member may translate in a linear direction relative to the housing support member. The medical device engagement member may slide in a linear direction relative to the housing support member. The medical device engagement member may be slidably engageable with the housing support member.

The mounting apparatus may include a longitudinal axis. The longitudinal axis may extend between a top portion and a bottom portion of the mounting apparatus.

The mounting apparatus may include a lateral axis. The lateral axis may extend between a left portion and a right portion of the mounting apparatus.

The medical device engagement member may translate in a direction which is parallel to the longitudinal axis of the mounting apparatus.
The medical device may be an X-ray tube. The medical device may be an X-ray tube head. The medical device may be a radiotherapy X-ray tube.

The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The housing support member may be configured to support the medical device engagement member. The housing support member may be configured to receive the medical device engagement member. The housing support member may be configured to receive and support the medical device engagement member. The housing support member may be configured to suspend the medical device engagement member.

The housing support member may be configured to provide vertical support to the medical device engagement member. The housing support member may be configured to provide horizontal support to the medical device engagement member. The housing support member may be configured to provide vertical and/or horizontal support to the medical device engagement member.

The housing support member may be configured to provide vertical support to the medical device engagement member via the adjustment device.

The housing support member may be pivotably attachable to a support arm of a medical device system. The housing support member may be pivotably attachable to an attachment member of a support arm of a medical device system. The housing support member may be pivotably attachable to a yoke member of a support arm of a medical device system.

The housing support member may be generally cylindrical in shape. The housing support member may be a hollow cylinder.

The housing support member may include a side wall. The side wall may be a substantially cylindrical wall.

The housing support member may include an upper, or top, portion, and a lower, or bottom, portion.

The housing support member may include a support structure attachment portion. The support structure attachment portion may be configured to allow the housing support member to be attachable to a support structure. The support structure attachment portion may be configured to allow the housing support member to be attachable to an attachment portion of a support structure. The support structure may be a support arm of a medical device system. The attachment portion of the support structure may be a yoke.

The housing support member may be attached to a support structure, or the attachment portion, by one or more mechanical fixing devices.

The housing support member may include one or more apertures in the side wall thereof. The housing support member may include two apertures in the side wall. The two apertures may be arranged to oppose one another. The two apertures may be located on opposite portions of the side wall. The two apertures may be oppositely facing. The two apertures may be substantially diametrically opposite from one another.

The, or each, aperture in the side wall of the housing support member may be an elongate slot. The two oppositely facing apertures in the side wall of the housing support member may be elongate slots. The, or each, aperture in the side wall of the housing support member may be an elongate channel. The two oppositely facing apertures in the side wall of the housing support member may be elongate channels.

The, or each, aperture in the side wall of the housing support member may extend in a circumferential direction. The, or each, aperture in the side wall of the housing support member may extend in a circumferential direction of the side wall.

The, or each, aperture in the side wall of the housing support member may extend about the longitudinal axis of the mounting apparatus.

The, or each, aperture in the side wall of the housing support member may lie on a plane. The plane may be orthogonal to the longitudinal axis of the mounting apparatus.

The, or each, aperture in the side wall of the housing support member may lie on a horizontal plane. That is, the, or each, aperture in the side wall of the housing support member extends only in the horizontal direction, or plane. That is, the, or each, aperture in the side wall of the housing support member extends only on the plane.

The, or each, aperture in the side wall of the housing support member may be a horizontal slot. The, or each, slot may be orthogonal to the longitudinal axis of the mounting apparatus. The, or each, aperture in the side wall of the housing support member may be a horizontal channel. The, or each, channel may be orthogonal to the longitudinal axis of the mounting apparatus.

Each aperture in the side wall of the housing support member may be substantially the same length. Each horizontal slot in the side wall of the housing support member may be substantially the same length. Each horizontal channel in the side wall of the housing support member may be substantially the same length.

The, or each, aperture in the side wall of the housing support member may be located between the top and bottom of the housing support member.

The housing support member may include an adjustment device support. The adjustment device support may be configured to provide support to the adjustment device. The adjustment device support may be configured to provide vertical support to the adjustment device.

The adjustment device support may be located on the lower portion of the housing support member. The adjustment device support may be located on the outwardly facing surface of the side wall of the housing support member. The adjustment device support may protrude from the outwardly facing surface of the side wall of the housing support member. The adjustment device support may protrude from the outwardly facing surface of the side wall of the housing support member. The adjustment device support may extend around the outwardly facing surface of the side wall of the housing support member. The adjustment device support may extend around substantially the entire outwardly facing surface of the side wall of the housing support member.

The adjustment device support may be a channel portion. The channel portion be configured to receive at least a portion of the adjustment device therein. The channel portion be configured to receive at least a portion of a lower portion of the adjustment device therein. The channel portion may extend around the outwardly facing surface of the side wall of the housing support member. The channel portion may extend around substantially the entire outwardly facing surface of the side wall of the housing support member.

The housing support member may include channel portion. The channel portion may be configured to receive at least a portion of the medical device engagement member. The channel portion may be configured to receive an attachment member, or attachment portion, of the medical device engagement member. The channel portion may be configured to receive an attachment member, or attachment portion, of the medical device engagement member, or the medical device. The channel portion may be an attachment member, or attachment portion, channel portion. The channel portion may be configured to allow relative movement between the housing support member and the medical device engagement member. The channel portion may be configured to allow linear translational movement between the housing support member and the medical device engagement member.

The medical device engagement member may be arranged to move in a linear direction relative to the housing support member. The medical device engagement member may be arranged to move relative to the housing support member in the direction of the longitudinal axis of the housing support member.

The medical device engagement member may be configured to be engageable with a medical device. The medical device engagement member may be configured to be detachably engageable with a medical device.

The medical device engagement member may be configured to receive a medical device. The medical device engagement member may be configured to receive and engage with a medical device.

The medical device engagement member may be configured to be secured to a medical device. The medical device engagement member may be configured to be secured to a medical device with one or more fastening devices. The fastening devices may be mechanical fastening devices.

The medical device engagement member may be configured to be secured to a medical device via an attachment member. The attachment member may be attachable to a medical device and the attachment member may be attachable to the medical device engagement member. The attachment member may be attachable to the medical device and the medical device engagement member with one or more fastening devices. The fastening devices may be mechanical fastening devices.

The medical device engagement member may be configured to clamp around a medical device. The medical device engagement member may be a clamp member.

The medical device engagement member may include an attachment portion. The attachment portion may be configured such that the medical device engagement member may be attachable to a medical device. The attachment portion may be configured such that the medical device engagement member may be attachable to an attachment member of a medical device.

The medical device engagement member may be generally cylindrical in shape. The medical device engagement member may be a hollow cylinder.

The medical device engagement member may include a side wall. The side wall may be a substantially cylindrical wall.

The medical device engagement member may include an upper, or top, portion, and a lower, or bottom, portion.

The medical device engagement member may include one or more apertures in the side wall thereof. The medical device engagement member may include two apertures in the side wall. The two apertures may be arranged to oppose one another. The two apertures may be located on opposite portions of the side wall. The two apertures may be oppositely facing. The two apertures may be substantially diametrically opposite from one another.

The, or each, aperture in the side wall of the medical device engagement member may be an elongate slot. The two oppositely facing apertures in the side wall of the medical device engagement member may be elongate slots. The, or each, aperture in the side wall of the medical device engagement member may be an elongate channel. The two oppositely facing apertures in the side wall of the medical device engagement member may be elongate channels.

The, or each, aperture in the side wall of the medical device engagement member may extend in a circumferential direction. The, or each, aperture in the side wall of the medical device engagement member may extend in a circumferential direction of the side wall. The, or each, aperture in the side wall of the medical device engagement member may extend in a spiralling circumferential direction. The, or each, aperture in the side wall of the medical device engagement member may extend in a spiralling circumferential direction of the side wall. That is, the, or each, aperture in the side wall takes the form of a partial helix.

The, or each, aperture in the side wall of the medical device engagement member may extend about the longitudinal axis of the mounting apparatus.

The, or each, aperture in the side wall of the medical device engagement member may be arranged to be inclined to a plane. That is, the, or each, aperture in the side wall of the medical device engagement member extends at an angle to the horizontal direction, or plane. The plane may be orthogonal to the longitudinal axis of the mounting apparatus.

The, or each, aperture in the side wall of the medical device engagement member may be arranged to be inclined to a horizontal plane. That is, the, or each, aperture in the side wall of the medical device engagement member extends at an angle to the horizontal direction, or plane. The plane may be orthogonal to the longitudinal axis of the mounting apparatus.

The, or each, aperture in the side wall of the medical device engagement member may be arranged to extend from the lower, or bottom, portion of the medical device engagement member towards the upper, or top, portion of the medical device engagement member. The, or each, aperture in the side wall of the medical device engagement member may be arranged to extend in the horizontal and vertical direction from the lower, or bottom, portion of the medical device engagement member towards the upper, or top, portion of the medical device engagement member. The, or each, aperture in the side wall of the medical device engagement member may be arranged to extend in the horizontal and vertical direction.

The, or each, aperture in the side wall of the medical device engagement member may include a contact surface. The contact surface may be configured to engage with a contact member. The contact surface may be a cammed surface.

The, or each, aperture in the side wall of the medical device engagement member may be a channel portion. The channel portion may be configured to receive a contact member therein. The channel portion may be configured to allow a contact member to progress from one end of the channel to the other.

The angle of inclination of the, or each, aperture may be between 6.5 degrees and 12.5 degrees from horizontal. The angle of inclination of the, or each, aperture may be 9.5 degrees.

Each aperture in the side wall of the medical device engagement member may be substantially the same length. Each horizontal slot in the side wall of the medical device engagement member may be substantially the same length. Each horizontal channel in the side wall of the medical device engagement member may be substantially the same length.

The, or each, aperture in the side wall of the medical device engagement member may be located between the top and bottom of the housing support member. The, or each, aperture in the side wall of the medical device engagement member may progress between the top and bottom of the housing support member as the aperture progresses about the circumference of the side wall.

The medical device engagement member may be configured to allow linear translational movement between the housing support member and the medical device engagement member.

The attachment portion of the medical device engagement member may include one or more low friction portions. The attachment portion of the medical device engagement member may include one or more slip pads. The one or more slip pads, or portions with relatively lower coefficient of friction values than the other surfaces of the attachment portion, are arranged to contact the channel portion of the housing support member.

The attachment portion and/or attachment member of the medical device engagement member may include one or more low friction portions. The attachment portion and/or the attachment member of the medical device engagement member may include one or more slip pads. The one or more slip pads, or portions with relatively lower coefficient of friction values than the other surfaces of the attachment portion, are arranged to contact the channel portion of the housing support member.

The housing support member may be configured to receive the medical device engagement member therein.

The housing support member and the medical device engagement member may substantially concentrically arranged. That is, the longitudinal axes of the housing support member and the medical device engagement member may be colinear with the longitudinal axis of the mounting apparatus.

The housing support member and the medical device engagement member may be arranged such that relative linear translational movement between the two is permitted.

The housing support member may be configured to receive the medical device engagement member therein, and allow the medical device engagement member to linearly move into and out of engagement with the housing support member in the direction of the longitudinal axis of the mounting apparatus.

The housing support member may be configured to receive the medical device engagement member therein, and allow the medical device engagement member to linearly move the housing support member in the direction of the longitudinal axis of the mounting apparatus.

The housing support member and the medical device engagement member may be arranged such that relative rotational movement about the longitudinal axis of the mounting apparatus between the two is prohibited. The engagement of the attachment portion, or attachment member, of the medical device engagement member in the channel portion of the housing support member may prohibit relative rotational movement between the housing support member and the medical device engagement member about the longitudinal axis of the mounting apparatus.

The housing support member and the medical device engagement member may be arranged such that relative linear translational movement between the two is permitted, and relative rotational movement about the longitudinal axis of the mounting apparatus between the two is prohibited.

The housing support member may be configured to receive the medical device engagement member therein, and allow the medical device engagement member to linearly move into and out of engagement with the housing support member in the direction of the longitudinal axis of the mounting apparatus, and prevent relative rotational movement about the longitudinal axis of the mounting apparatus.

The outer diameter of the medical device engagement member may be less than the inner diameter of the housing support member.

The housing support member and the medical device engagement member may be arranged such that the outwardly facing surface of the side wall of the medical device engagement member faces the inwardly facing surface of the side wall of the housing support member. In this arrangement a gap may exist between the outwardly facing surface of the side wall of the medical device engagement member and the inwardly facing surface of the side wall of the housing support member. This gap may be filled with a sleeve member. The sleeve member may be a low friction member. The sleeve member may be located between the housing support member and the medical device engagement member. The sleeve member may be attachable to the adjustment device. The sleeve member may have a relatively lower coefficient of friction value than the other surfaces of the housing support member or medical device engagement member.

The housing support member and the medical device engagement member may be arranged such that their respective apertures face one another. That is, the, or each, aperture on the side wall of the housing support member faces the, or each, aperture on the side wall of the medical device engagement member.

The adjustment device may be configured to move the medical device engagement member relative to the housing support member.

The adjustment device may be configured to move the medical device engagement member between a first position and a second position. The first position may be an initial position and the second position may be an extended position. The first position may be a minimum extension position and the second position may be a maximum extended position.

The adjustment device may be configured to move the medical device engagement member in a linear direction relative to the housing support member. The adjustment device may be configured to move the medical device engagement member in the direction of the longitudinal axis of the housing support member. The adjustment device may be configured to move the medical device engagement member into and out of engagement with the housing support member. The adjustment device may be configured to move the medical device engagement member linearly in the direction of the longitudinal axis of the mounting apparatus.

The arrangement of the housing support member, the medical device engagement member and the adjustment device is such that the medical device engagement member may move a distance of between 5 mm and 15 mm along the direction of the longitudinal axis of the mounting apparatus. The arrangement of the housing support member, the medical device engagement member and the adjustment device is such that the medical device engagement member may move a distance of 10 mm along the direction of the longitudinal axis of the mounting apparatus.

The adjustment device may include a body portion. The body portion may be rotatably engageable with the housing support member. The body portion may be configured to engage with the housing support member and rotate about the longitudinal axis of the mounting apparatus. The body portion may be configured to move between a first position and a second position. The first position of the body portion may correspond to the first position of the medical device engagement member, and the second position of the body portion may correspond to the second position of the medical device engagement member.

The body portion may be a collar member. The body portion may be a partial collar member. The body portion may be a wheel member. The body portion may be a partial wheel member.

The body portion may be a partial cylinder. The body portion may be partially cylindrical in shape. The body portion may be a partial hollow cylinder.

The body portion may include a side wall. The side wall may be partially cylindrical in shape.

The body portion may include an upper, or top, portion, and a lower, or bottom, portion.

The lower, or bottom, portion of the side wall of the body portion may be engageable with the adjustment device support of the housing support member. The side wall of the body portion may be engageable with the channel portion of the adjustment device support of the housing support member.

The adjustment device support of the housing support member may be configured to provide support to the body portion of the adjustment device. The adjustment device support of the housing support member may be configured to provide support to the body portion of the adjustment device between its first position and its second position.

The adjustment device may include one or more medical device engagement member engaging members. The engaging members may be configured to engage the medical device engagement member via the one or more apertures in the side wall of the medical device engagement member. The engaging members may be configured to engage the medical device engagement member via the one or more apertures in the side wall of the housing support member. The engaging members may be configured to engage the apertures of the medical device engagement member via the apertures in the side wall of the housing support member.

The engaging members may protrude from an inwardly facing surface of the side wall of the adjustment device. The engaging members may protrude from the inwardly facing surface of the side wall of the adjustment device, through the one or more apertures in the side wall of the housing support member and into the one or more apertures in the side wall of the medical device engagement member.

The engaging members may be arranged to engage with the elongate slots in the side wall of the housing support member and the elongate slots in the side wall of the medical device engagement member.

In this arrangement, as the engaging members of the adjustment device protrude through the elongate slots in the side wall of the housing support member to engage with the elongate slots in the side wall of the medical device engagement member, rotation of the adjustment device relative to the housing support member from the first position to the second position causes the medical device engagement member to move between its first position and its second position.

In this arrangement, as the elongate slots in the side walls of the housing support member and the medical device engagement member are angularly offset from one another, the engaging members of the adjustment device engage the contact surface of the elongate slot of the medical device engagement member and exert a force in the direction of the longitudinal axis of the mounting apparatus as the body portion of the adjustment device is rotated relative to the housing support member.

The engaging members of the adjustment device may be protruding members. The engaging members of the adjustment device may be pin members. The engaging members may be removably attachable to the body portion of the adjustment device. The engaging members may be screwed into the body portion of the adjustment device. The pin members may be held in place on the body portion by one or more fixing members. The engaging members of the adjustment device may be cylindrical members. The engaging members of the adjustment device may be cylindrical members. The engaging members of the adjustment device may be cylindrical protrusions.

The engaging members of the body portion of the adjustment device may be positioned opposite one another. The engaging members of the body portion of the adjustment device may be substantially diametrically opposed.

The body portion of the adjustment device may include one or more gripping portions. The gripping portions may include one or more protrusions. The gripping portions may be thumb grips. The gripping portions may aid in turning of the adjustment device. The gripping portions may aid in the rotation of the adjustment device relative to the housing support member.

The body portion may include a lever member. The lever member may be a handle. The lever member may project from the body portion of the adjustment device. The lever member may be pivotably connected to the body portion. The lever member may aid in turning of the adjustment device. The lever member may aid in the rotation of the adjustment device relative to the housing support member.

The mounting apparatus may further comprise a medical device. The medical device may be engaged with the medical device engagement member.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The mounting apparatus may further comprise a support structure. The support structure may be a support structure of a medical device system. The support structure may be a support arm of a medical device system. The mounting apparatus may be attached to the support structure.

The mounting apparatus may further comprise a base unit. The base unit may provide support to the support structure. The support structure may be attached to the base unit.

The base unit may include one or more wheels, castors, or the like, to allow movement of the base unit.

According to a second aspect of the present invention there is provided a support structure for a medical device system, comprising:
a mounting apparatus for a medical device, comprising:
   a housing support member;
   a medical device engagement member, the medical device engagement member being moveably engageable with the housing support member; and
   an adjustment device, the adjustment device being configured to move the medical device engagement member relative to the housing support member.

The support structure may be a support arm. The support structure may be a moveable arm.

The mounting apparatus may further comprise a medical device. The medical device may be engaged with the medical device engagement member.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The support structure may further comprise a base unit. The base unit may provide support to the support structure. The support structure may be attached to the base unit.

The base unit may include one or more wheels, castors, or the like, to allow movement of the base unit.

The medical device system may be an X-ray system. The medical device system may be a radiotherapy X-ray system.

Embodiments of the second aspect of the present invention may include one or more features of the first aspect of the present invention or its embodiments. Similarly, embodiments of the first aspect of the present invention may include one or more features of the second aspect of the present invention or its embodiments.

According to a third aspect of the present invention there is provided a medical device system, comprising:
a base unit; and
a support structure for supporting a medical device mounting apparatus, the support structure being attachable to the base unit; and
a mounting apparatus for a medical device, comprising:
   a housing support member;
   a medical device engagement member, the medical device engagement member being moveably engageable with the housing support member; and
   an adjustment device, the adjustment device being configured to move the medical device engagement member relative to the housing support member.

The base unit may provide support to the support structure. The support structure may be attached to the base unit.

The base unit may include one or more wheels, castors, or the like, to allow movement of the base unit.

The support structure may be a support arm. The support structure may be a moveable arm.

The medical device system may further comprise a medical device. The medical device may be engaged with the medical device engagement member.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The medical device system may be an X-ray system. The medical device system may be a radiotherapy X-ray system.

Embodiments of the third aspect of the present invention may include one or more features of the first or second aspects of the present invention or their embodiments. Similarly, embodiments of the first or second aspects of the present invention may include one or more features of the third aspect of the present invention or its embodiments.

According to a fourth aspect of the present invention there is provided a medical device system, comprising:
a base unit;
a support structure for supporting a medical device mounting apparatus, the support structure being attachable to the base unit; and
a mounting apparatus for a medical device, comprising:
   a housing support member;
   a medical device engagement member, the medical device engagement member being moveably engageable with the housing support member;
   an adjustment device, the adjustment device being configured to move the medical device engagement member relative to the housing support member; and
a medical device mounted to the mounting apparatus.

The base unit may provide support to the support structure. The support structure may be attached to the base unit.

The base unit may include one or more wheels, castors, or the like, to allow movement of the base unit.

The support structure may be a support arm. The support structure may be a moveable arm.

The medical device may be engaged with the medical device engagement member.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The medical device system may be an X-ray system. The medical device system may be a radiotherapy X-ray system.

Embodiments of the fourth aspect of the present invention may include one or more features of the first, second or third aspects of the present invention or their embodiments. Similarly, embodiments of the first, second or third aspects of the present invention may include one or more features of the fourth aspect of the present invention or its embodiments.

According to a fifth aspect of the present invention there is provided a mounting apparatus for a device, comprising:
a support member;
a device engagement member, the device engagement member being moveably engageable with the support member; and
an adjustment device, the adjustment device being configured to move the device engagement member relative to the support member.

The device may be a medical device.

The support member may be a housing support member.

Embodiments of the fifth aspect of the present invention may include one or more features of the first, second, third, or fourth aspects of the present invention or their embodiments. Similarly, embodiments of the first, second, third, or fourth aspects of the present invention may include one or more features of the fifth aspect of the present invention or its embodiments.

According to a sixth aspect of the present invention there is provided a method of adjusting the position of a medical device engagement member of a mounting apparatus for a medical device, comprising the steps of:
providing a mounting apparatus for a medical device, comprising:
   a housing support member;
   a medical device engagement member, the medical device engagement member being moveably engageable with the housing support member; and
   an adjustment device, the adjustment device being configured to move the medical device engagement member relative to the housing support member; and
   adjusting the position of the medical device engagement member by using the adjustment device to move the medical device engagement member relative to the housing support member.

The mounting apparatus may further comprise a medical device.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

Embodiments of the sixth aspect of the present invention may include one or more features of the first, second, third, fourth, or fifth aspects of the present invention or their embodiments. Similarly, embodiments of the first, second, third, fourth, or fifth aspects of the present invention may include one or more features of the sixth aspect of the present invention or its embodiments.

According to a seventh aspect of the present invention there is provided a device support system, comprising:
a base unit;
a support structure for supporting a device mounting apparatus, the support structure being attachable to the base unit; and
a mounting apparatus for a device, comprising:
   a support member;
   a device engagement member, the medical device engagement member being moveably engageable with the housing support member; and
   an adjustment device, the adjustment device being configured to move the medical device engagement member relative to the housing support member.

The base unit may provide support to the support structure. The support structure may be attached to the base unit.

The base unit may include one or more wheels, castors, or the like, to allow movement of the base unit.

The support structure may be a support arm. The support structure may be a moveable arm.

The device support system may further comprise a device. The device may be engaged with the device engagement member.

Embodiments of the seventh aspect of the present invention may include one or more features of the first, second, third, fourth, fifth, or sixth aspects of the present invention or their embodiments. Similarly, embodiments of the first, second, third, fourth, fifth, or sixth aspects of the present invention may include one or more features of the seventh aspect of the present invention or its embodiments.

### BRIEF DESCRIPTION OF THE DRAWINGS

Embodiments of the invention will now be described, by way of example, with reference to the drawings, in which:

FIG. 1a is a perspective view of a mounting apparatus for a medical device according to the present invention with a medical device mounted thereon;

FIG. 1b is an alternative perspective view of the mounting apparatus of FIG. 1a;

FIG. 1c is a top view of the mounting apparatus of FIG. 1a;

FIG. 1d is a perspective view of the mounting apparatus of FIG. 1a showing detail of the medical device engagement member;

FIG. 1e is a partial cut-away top view of the mounting apparatus of FIG. 1a without the medical device;

FIG. 1f is a perspective partial cut-away side view of the mounting apparatus of FIG. 1a without the medical device;

FIG. 2 is an exploded perspective view of the mounting apparatus of FIG. 1a;

FIGS. 3a to 3c are side views of the mounting apparatus of FIG. 1a illustrating different positions of the adjustment device;

FIGS. 3d to 3f are top perspective views of the mounting apparatus of FIG. 1a illustrating different positions of the adjustment device; and

FIG. 4 is a schematic view of a medical device system according to the present invention.

### DESCRIPTION OF PREFERRED EMBODIMENTS

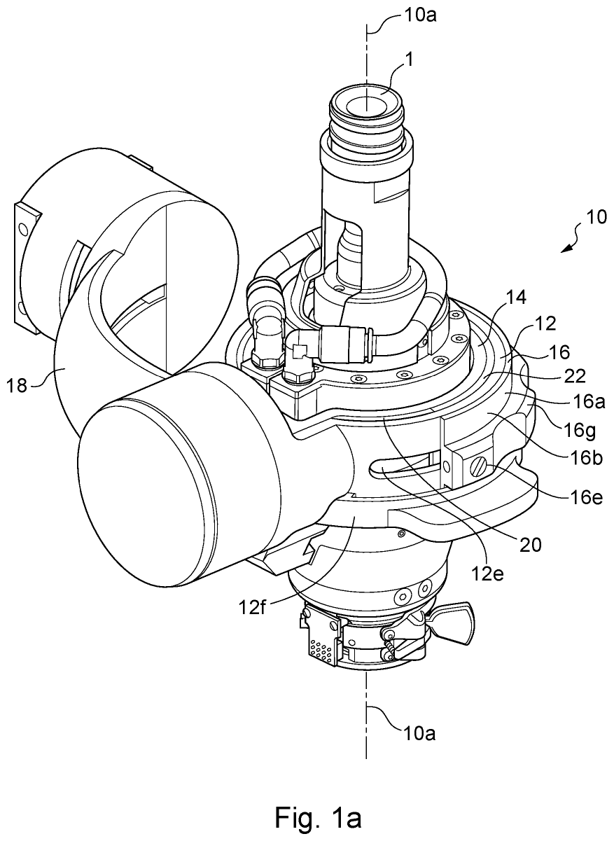

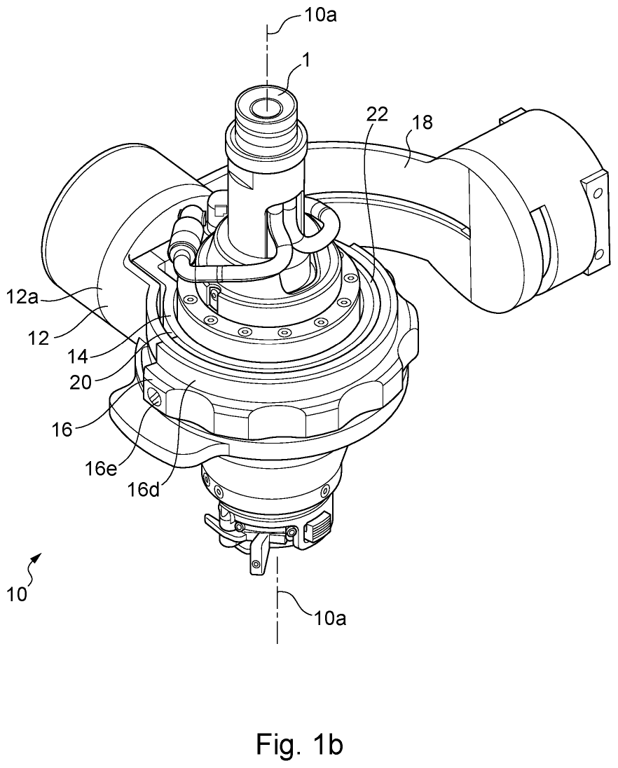

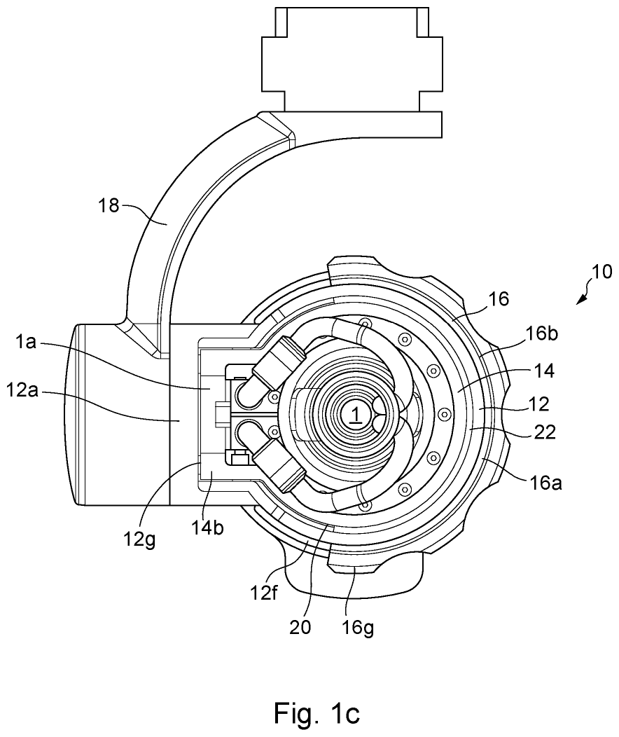

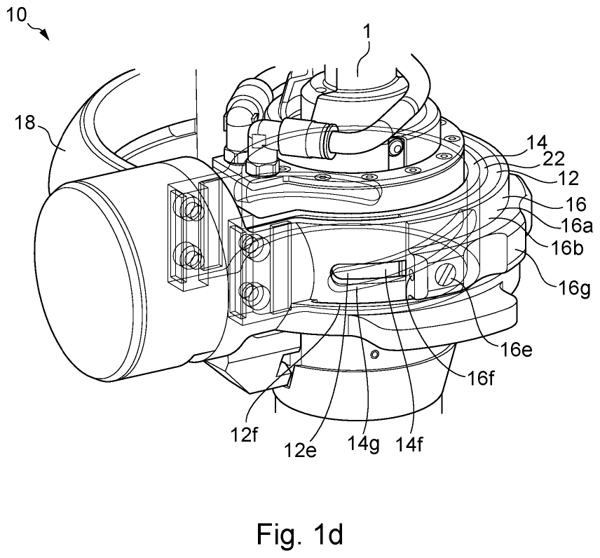

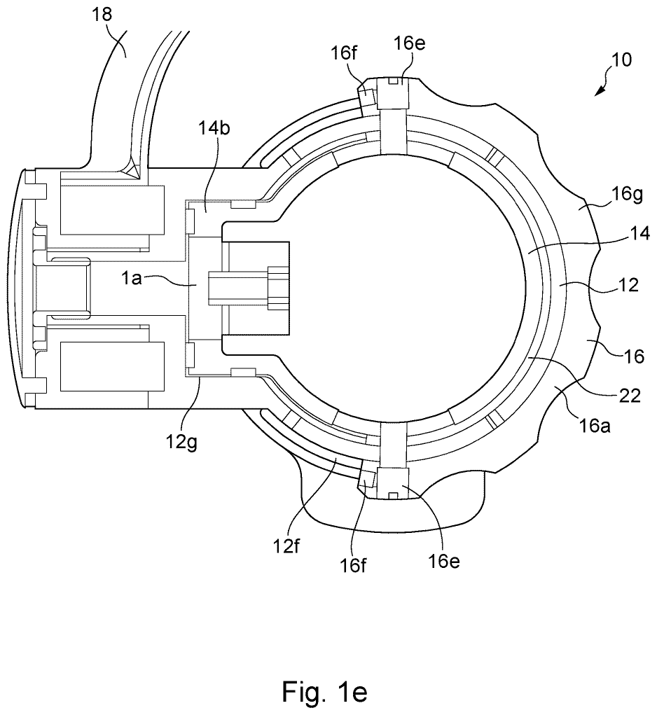

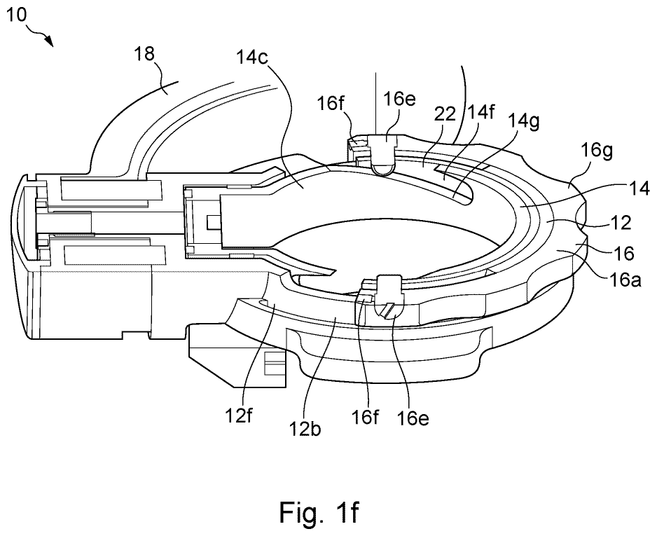

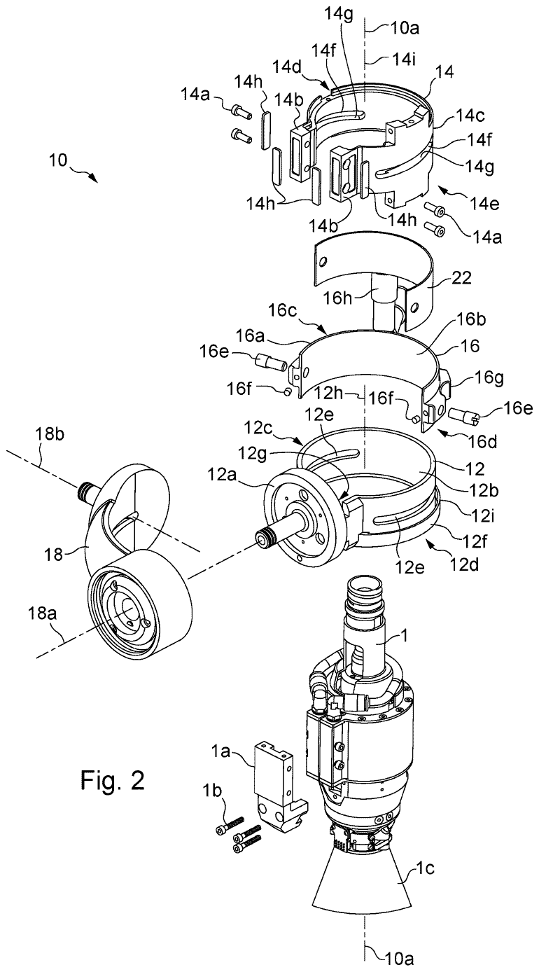

With reference to FIGS. 1 and 2, a mounting apparatus for a medical device 10 is illustrated. The mounting apparatus 10 includes a housing support member 12 (an example of a support member), a medical device engagement member 14 (an example of an engagement member), and an adjustment device 16.

In the embodiment illustrated and described here, the mounting apparatus 10 is used to mount an X-ray tube head 1. However, it should be appreciated that the mounting apparatus 10 may be used to mount other types of devices and medical devices.

The X-ray tube head 1 may be a radiotherapy X-ray tube for the treatment of skin conditions, such as malignant or benign skin cancers or superficial skin lesions. In the embodiment illustrated and described here, the X-ray tube 1 is an end-window X-ray tube. The X-ray tube head 1 also includes an applicator 1c (see FIG. 2). The applicator 1c contacts the skin of a patient in use to ensure that a predetermined distance exists between the X-ray tube head 1 and the patient's skin. The applicator 1c also exerts a pressure on the patient's skin to flatten the area to be exposed to the X-rays. In the embodiment illustrated and described here, the applicator 1c is a frusto-conical member made of Perspex (i.e., Poly(methyl methacrylate) or PMMA). However, it should be appreciated that other shapes or materials may be used.

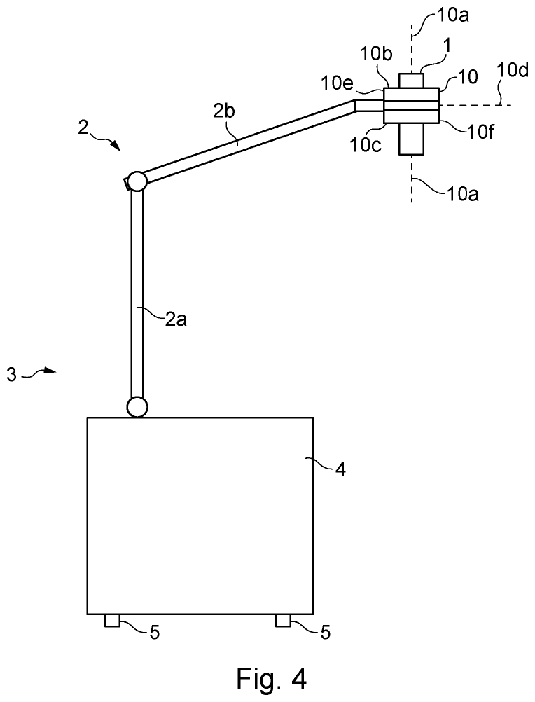

As illustrated in FIG. 4, the mounting apparatus 10 and X-ray tube head 1 (an example of a device) may be mounted to a support arm 2 (an example of a support structure) of a medical device system 3 (an example of a device support system). The medical device system 3 includes a base unit 4 upon which the support arm 2 is mounted. The base unit 4 includes wheels 5 in order to transport the system 3.

The base unit 4 allows the system 3 to be moved into position adjacent a patient (not shown) during use, and the support arm 2 may be manipulated into position such that the X-ray tube head 1 can be located close to the portion of the patient's skin to be treated. The support arm 2 may be pivotably and rotatably connected to the base unit 4. The support arm 2 includes a first arm member 2a and a second arm member 2b. The first arm member 2a and the second arm member 2b are pivotably connected to one another. The mounting apparatus 10 is pivotably attachable to the second arm member 2b of the support arm 2. The first arm member 2a is pivotably attachable to the base unit 4 of the system 3. As described further below, the mounting apparatus 10 may be attached directly to the support arm 2, or indirectly attached to the support arm 2 by an attachment member, or yoke member, 18 of the support arm 2. The system 3 includes a number of brake devices (not shown) to hold the support arm 2 in position.

With reference to FIGS. 1 and 2, the mounting apparatus 10 includes a longitudinal axis 10a, which extends between a top, or upper, portion 10b of the mounting apparatus 10, and a bottom, or lower, portion 10c of the mounting apparatus 10 (see FIG. 4). The mounting portion 10 also includes a lateral axis 10d, which extends between a left portion, or left side portion 10e, and a right portion, or right side portion 10f of the mounting apparatus 10 (see FIG. 4).

As described further below, the mounting apparatus 10 engages with, and provides adjustable support to, the X-ray tube head 1. The mounting apparatus 10 principally provides vertical support to the X-ray tube head 1. As illustrated, in the embodiment illustrated and described here, the mounting apparatus 10 may pivot in at least two orthogonal axes 18a, 18b with respect to the support arm 2 (see FIG. 2).

As illustrated, the housing support member 12 is configured to receive and support the medical device engagement member 14 therein. As described further below, the housing support member 12 supports the medical device engagement member 14 via the adjustment device 16.

The housing support member 12 includes a support structure attachment portion 12a which is used to attach the housing support member 12 to the support arm 2. In the embodiment illustrated and described here, the support structure attachment portion 12a attaches to the attachment member 18 of the support arm 2. The mounting apparatus 10 may pivot about a first axis 18a relative to the attachment member 18, and the attachment member 18 may pivot about a second axis 18b relative to the support arm 2. Axes 18a and 18b are orthogonal to one another. However, it should be appreciated that the housing support member 12 may be directly connected to the support arm 2, or via another type of pivotable connection, such as a universal ball joint, or the like. The housing support member 12 may be attached to the support arm 2 by one or more mechanical fixing devices (not show), or the like.

The housing support member 12 is generally cylindrical in shape and has a cylindrical side wall 12b. The housing support member 12 has an upper, or top, portion 12c, and a bottom, or lower, portion 12d (see FIG. 2).

As best illustrated in FIGS. 2 and 1a, the housing support member 12 includes two oppositely facing apertures 12e located in the side wall 12b thereof. The apertures 12e are elongated slots, or channels, that extend in the circumferential direction of the side wall 12b. As illustrated, the apertures 12e extend about the longitudinal axis 10a of the mounting apparatus 10 on a horizontal plane. The horizontal plane is orthogonal to the longitudinal axis 10a of the mounting apparatus 10. That is, the apertures 12e extend only in the horizontal direction, or plane. Each aperture 12e is substantially the same length. In the embodiment illustrated in described here, each aperture 12e is located approximately between the upper portion 12c and the lower portion 12d of the side wall 12b.

As best illustrated in FIGS. 1a, 1f and 2, and described further below, the housing support member 12 includes an adjustment device support 12f. The adjustment device support 12f is configured receive a lower portion of the adjustment device 16. In the embodiment illustrated and described here, the adjustment device support 12f is a circumferential channel 12i that protrudes from the lower portion 12d of the side wall 12b.

As best illustrated in FIGS. 1a, 1c, 1e and 1f, and described further below, the housing support member 12 includes a channel portion 12g, which is configured to receive at least an attachment portion 14b of the medical device engagement member 14. As described further below, the channel portion 12g is arranged to allow the medical device engagement member 14 to slide vertically with respect to the housing support member 12. In this arrangement, the medical device engagement member 14 is permitted to move in the direction of the longitudinal axis 10a of the mounting apparatus 10.

As described above, the medical device engagement member 14 is configured to receive and engage with a medical device 1. The X-ray tube head 1 may be detachable engageable with the medical device engagement member 14. The medical device engagement member 14 is moveably engageable with the housing support member 12.

With reference to FIG. 2, in the embodiment illustrated and described here, the X-ray tube head 1 may be attached to an attachment member 1a by a plurality of fastening devices 1b, and the attachment member 1a (and X-ray tube head 1) may be attached to the attachment portion 14b of the medical device engagement member 14 with a plurality of fastening devices 14a. However, it should be appreciated that, depending on the arrangement of the X-ray tube head 1 used, the attachment member 1a may not be required, or a different arrangement of attachment member 1a would be used.

In this arrangement, the medical device engagement member 14 clamps around the X-ray tube head 1.

The medical device engagement member 14 is generally cylindrical in shape and has a cylindrical side wall 14c. The medical device engagement member 14 has an upper, or top, portion 14d, and a bottom, or lower, portion 14e (see FIG. 2).

As best illustrated in FIGS. 1d, 1f and 2, the medical device engagement member 14 includes two oppositely facing apertures 14f located in the side wall 14c thereof. The apertures 14f are elongated slots, or channels, that extend in a spiralling circumferential direction of the side wall 14c. In this arrangement, the apertures 14f take the form of a partial helix. The partial helix may extend about the longitudinal axis 10a of the mounting apparatus 10. Each aperture 14f may be substantially the same length.

As illustrated, the apertures 14f extend about the longitudinal axis 10a of the mounting apparatus 10 at an angle to the horizontal plane. The horizontal plane is orthogonal to the longitudinal axis 10a of the mounting apparatus 10. That is, the apertures 14f extend in the horizontal and vertical direction. Each aperture 14f is substantially the same length. In the embodiment illustrated in described here, each aperture 14f extends from the lower, or bottom, portion 14e of the medical device engagement member 14 towards the upper, or top, portion 14d of the medical device engagement member 14.

As described further below, each aperture 14f in the side wall 14c of the medical device engagement member 14 includes a contact surface 14g. The contact surfaces 14g are configured to engage with a medical device engagement member engaging member (pin member 16e) of the adjustment device 16. In this arrangement, the apertures 14f may be considered channel portions, through which the pin members 16e may travel.

In the embodiment illustrated and described here, the angle of inclination that the apertures 14f make from the horizontal plane may be 9.5 degrees. Providing an angle of inclination of between X and Y ensures that the X-ray tube head 1 does not move downwards under gravity of its own weight.

As described further below, the medical device engagement member 14 is configured to allow linear translational movement between the housing support member 12 and the medical device engagement member 14. The attachment portion 14b of the medical device engagement member 14 includes slip pads 14h (an example of low friction portions, or portions with relatively lower coefficient of friction values than the other surfaces of the attachment portion 14b). The slip pads 14h aid in the sliding engagement between the medical device engagement member 14 and the housing support member 12.

As best illustrated in FIGS. 1a to 1d, the housing support member 12 is configured to receive the medical device engagement member 14 therein. In this arrangement, the housing support member 12 and the medical device engagement member 14 are substantially concentrically arranged. That is, the longitudinal axis 12h of the housing support member and the longitudinal axis 14i of the medical device engagement member 14 may be colinear with the longitudinal axis 10a of the mounting apparatus 10 (see FIG. 2. Note: the longitudinal axis 10a of the mounting apparatus 10 is illustrated as being colinear with the longitudinal axis 14i of the medical device engagement member 14).

As discussed above, and will be appreciated from FIGS. 1 to 2, the housing support member 12 and the medical device engagement member 14 are arranged such that relative linear translational movement between the two is permitted, and relative rotational movement about the longitudinal axis 10a of the mounting apparatus 10 between the two is prohibited. The engagement of the attachment portion 14b of the medical device engagement member 14 in the channel portion 12g of the housing support member 12 prohibits relative rotational movement between the housing support member 12 and the medical device engagement member 14 about the longitudinal axis 10a of the mounting apparatus 10.

As best illustrated in FIG. 1c, the outer diameter of the medical device engagement member 14 may be less than the inner diameter of the housing support member 12, and the housing support member 12 and the medical device engagement member 14 are arranged such that the outwardly facing surface of the side wall 14c of the medical device engagement member 14 faces the inwardly facing surface of the side wall 12b of the housing support member 12. In this arrangement a gap 20 exists between the medical device engagement member 14 and the housing support member 12. This gap 20 may be filled with a sleeve member 22. The sleeve member 22 is a low friction member and is attachable to the adjustment device 16. The sleeve member 22 has a relatively lower coefficient of friction value than the other surfaces of the housing support member 12 or medical device engagement member 14, and aids in the sliding engagement between the medical device engagement member 14 and the housing support member 12.

As best illustrated in FIG. 1f, the housing support member 12 and the medical device engagement member 14 are arranged such that their respective apertures 12e and 14f face one another. That is, each aperture slot 12e on the side wall 12b of the housing support member 12 faces the aperture slot 14f on the side wall 14c of the medical device engagement member 14.

As described above, the adjustment device 16 is configured to move the medical device engagement member 14 relative to the housing support member 12. As described further below, the adjustment device 16 is configured to move the medical device engagement member 14 between a first position and a second position. The first position may be an initial position and the second position may be an extended position. The first position may be a minimum extension position and the second position may be a maximum extended position.

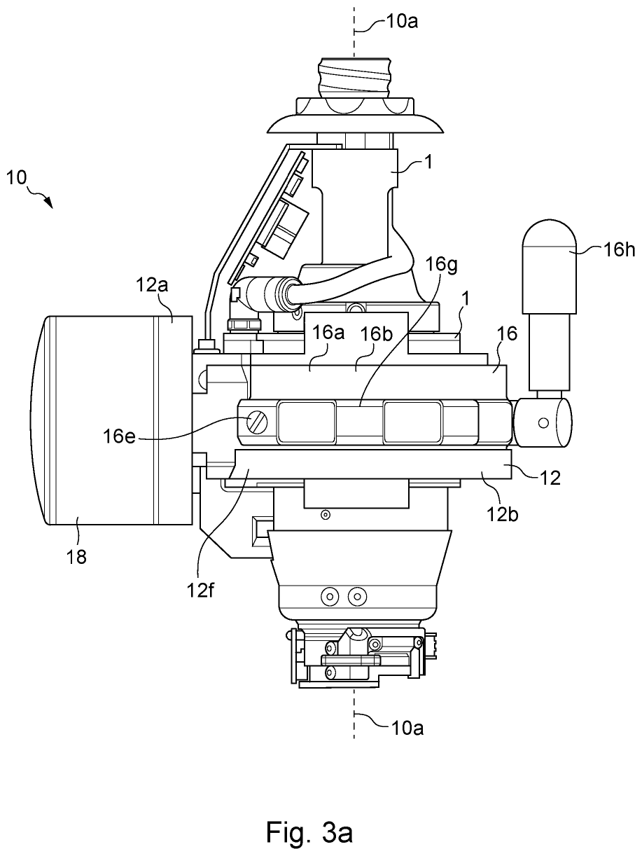

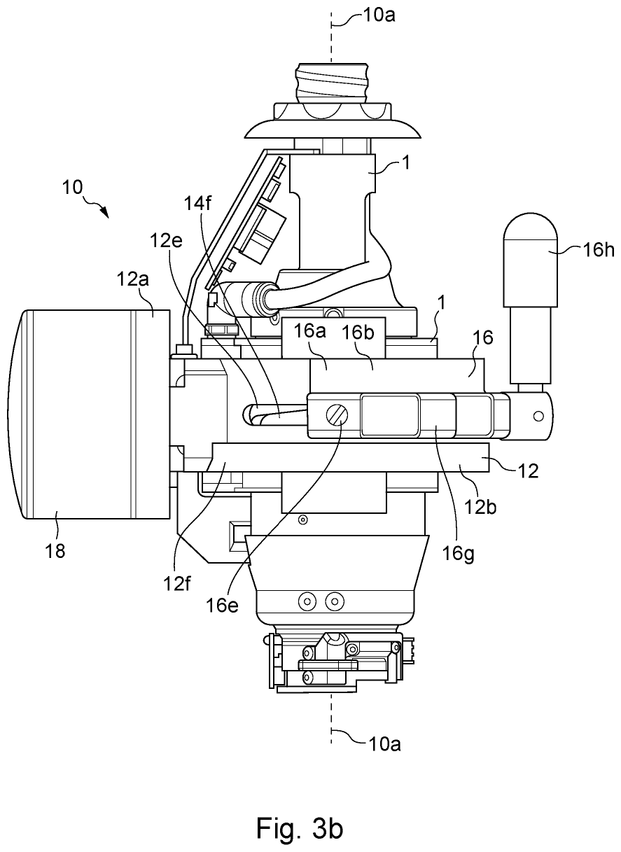

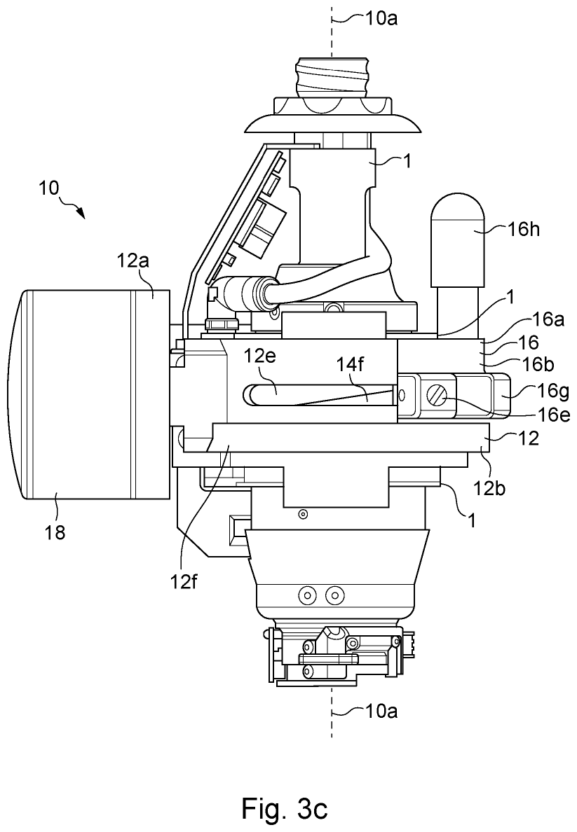

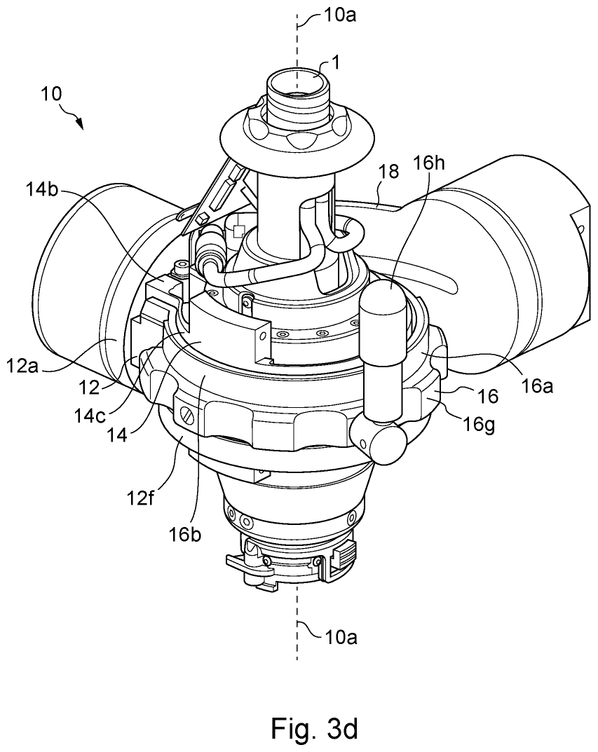

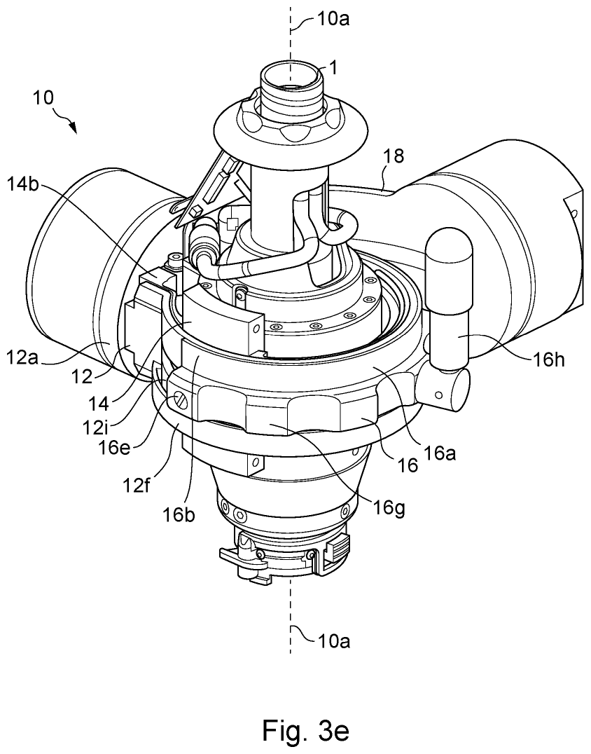

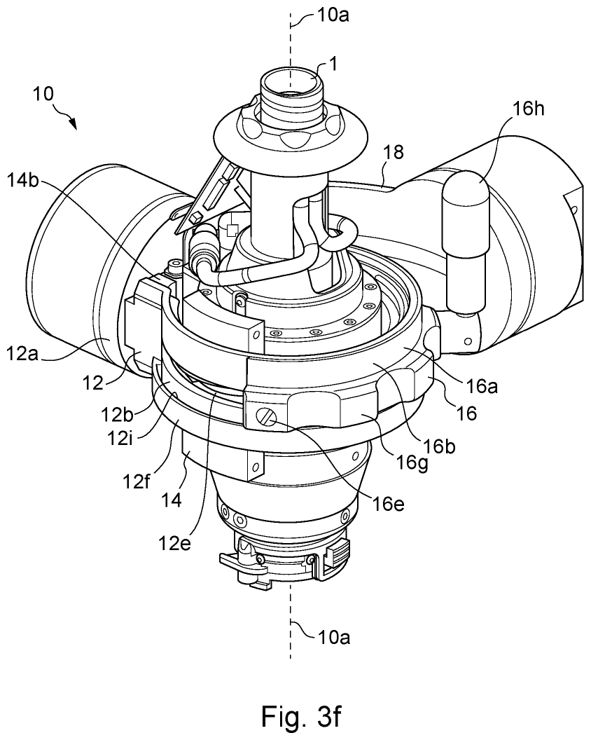

With reference to FIGS. 3a to 3c, FIG. 3a shows the first position of the medical device engagement member 14, FIG. 3b shows a middle position of the medical device engagement member 14 between the first position and the second position, and FIG. 3c shows the second position of the medical device engagement member 14.

The adjustment device 16 includes a body portion 16a, the body portion 16a being rotatably engageable with the housing support member 12 and configured to rotate about the longitudinal axis 10a of the mounting apparatus 10. The body portion 16a is configured to move between a first position (see FIG. 3a) and a second position (see FIG. 3c). The first position of the body portion 16a may correspond to the first position of the medical device engagement member 14, and the second position of the body portion 16a may correspond to the second position of the medical device engagement member 14.

In the embodiment illustrated and described here, the body portion 16a is a partial collar member with a partially cylindrical side wall 16b. The body portion 16a includes an upper, or top, portion 16c and a lower, or bottom, portion 16d (see FIG. 2).

The lower, or bottom, portion 16d of the side wall 16b of the body portion 16a is engageable with the adjustment device support 12f of the housing support member 12. In this arrangement, the side wall 16b of the body portion 16a is located in the channel defined by the adjustment device support 12f. As described above, the adjustment device support 12f of the housing support member 12 is configured to provide support to the body portion 16a of the adjustment device 16 between its first position and its second position.

As best illustrated in FIGS. 1e, 1f and 2, the adjustment device 16 includes two medical device engagement member engaging members 16e. In the embodiment illustrated and described here the medical device engagement member engaging members 16e are pin members. However, it should be appreciated that other types and shapes of engaging members may be used.

As described further below, the pin members 16e are configured to engage the apertures 14f of the medical device engagement member 14 via the apertures 12e in the side wall 12b of the housing support member 12.

The pin members 16e are attached to the body portion 16a and protrude from an inwardly facing surface of the side wall 16b of the body portion 16a. The pin members 16e protrude through the apertures 12e of the housing support member 12 and into the apertures 14f of the medical device engagement member 14. As described above, the apertures 12e and 14e are elongate slots, or channels.

In this arrangement, as the elongate slots 12e, 14f in the side walls 12b, 14c of the housing support member 12 and the medical device engagement member 14 are angularly offset from one another, the pin members 16e of the adjustment device 16 engage the contact surface 14g of the elongate slot 14f of the medical device engagement member 14 and exert a force in the direction of the longitudinal axis 10a of the mounting apparatus 10 as the body portion 16a of the adjustment device 16 is rotated relative to the housing support member 12. As describe further below, rotation of the adjustment device 16 relative to the housing support member 12 from the first position to the second position causes the medical device engagement member 14 to move between its first position and its second position. In the embodiment illustrated and described here, the pin members 16e slide on the contact surface 14g. However, it should be appreciated that other suitable types of engaging members could be used.

The pin members 16e may be removably attachable to the body portion 16a. In the embodiment illustrated and described here, the pin members 16e are attached to the body portion 16a by fastening devices 16f.

As illustrated, the pin members 16e are located substantially diametrically opposite one another across the body portion 16a.

The body portion 16a of the adjustment device 16 includes a plurality of gripping portions 16g. The gripping portions 16g being protruding portions that aid in the rotation of the adjustment device 16 by the thumb or hand or a user. The body portion 16a also includes a lever member 16h attached to the outer surface of the body portion 16a. The lever member 16h may be pivotably attached to the body portion 16a. Note: the lever member 16h is illustrated in FIG. 2 in a stowed position, and in FIGS. 3a to 3f in the deployed position. It may be pivoted downwards to be used by a user. Again, the lever member 16h may aid in the rotation of the adjustment device 16.

With reference to FIG. 2, the mounting apparatus 10 and X-ray tube head 1 are assembled together as follows. The X-ray tube head 1 is attached to the attachment member 1a by fastening devices 1b. The X-ray tube head 1 and attachment member 1a are then mounted to the attachment portion 14b of the medical device engagement member 14 by fastening devices 14a.

The X-ray tube head 1 and medical device engagement member 14 are then attached to the housing support member 12. This is carried out by (i) inserting the X-ray tube head 1 and medical device engagement member 14 into the housing support member 12, (ii) mounting the adjustment device 16 on to the housing support member 12, (iii) inserting the pin members 16e through the body portion 16a of the adjustment device 16, the apertures 12e in the housing support member 12, and the apertures 14f of the medical device engagement member 14, and (iv) securing the pin members 16e in place with the fastening devices 16f. In this arrangement, the housing support member 12 and the adjustment device 16 supports the X-ray tube head 1 and medical device engagement member 14.

With reference to FIGS. 3a to 3f, the operation of the mounting apparatus 10 will now be described. As described above, the adjustment device 16 is used raise and lower the X-ray tube head 1 relative to the housing support member 12. In this operation, the X-ray tube head 1 translates linearly in the direction of the longitudinal axis 10a of the mounting apparatus 10.

FIGS. 3a and 3d illustrate a first, or initial, position of the X-ray tube head 1, where it is at its highest vertical position, relative to the housing support member 12. FIGS. 3b and 3e illustrate the position where the body portion 16a of the adjustment device 16 has been partially rotated relative to the housing support member 12. As illustrated, in this position the X-ray tube head 1 is at a position between its highest vertical position, and lowest vertical position, relative to the housing support member 12. FIGS. 3c and 3f illustrate the position where the body portion 16a of the adjustment device 16 has been fully rotated relative to the housing support member 12. In this position, the X-ray tube head 1 is at its lowest vertical position, relative to the housing support member 12.

As described above, since the elongate slots 12e, 14f in the side walls 12b, 14c of the housing support member 12 and the medical device engagement member 14 are angularly offset from one another, the pin members 16e of the adjustment device 16 engage the contact surface 14g of the elongate slot 14f of the medical device engagement member 14 and exert a force in the direction of the longitudinal axis 10a of the mounting apparatus 10 as the body portion 16a of the adjustment device 16 is rotated relative to the housing support member 12. The rotation of the adjustment device 16 relative to the housing support member 12 from the first position to the second position causes the X-ray tube head 1 move between its first position and its second position, i.e., between its highest position and its lowest position, relative to the housing support member 12.

As illustrated, the body portion 16a of the adjustment device 16 rotates approximately 180 degrees between its starting position and end position.

It will be appreciated that rotating the body portion 16a back to its starting position moves the X-ray tube head 1 back to its highest vertical position from its lowest vertical position.

The arrangement of the housing support member 12, the medical device engagement member 14 and the adjustment device 16 is such that the X-ray tube head 1 may move a distance of 10 mm along the direction of the longitudinal axis 10a of the mounting apparatus 10.

Providing a mounting apparatus 10 for a medical device 1 as described above allows movement of the X-ray tube head 1 relative to the mounting apparatus 10, such that the X-ray tube head 1 can be moved closer to the patient once the base unit 4 and the support arm 2 have positioned the X-ray tube head 1 adjacent the portion of the patient's skin to be treated.

As described above, in order to deliver X-rays safely and accurately to a patient's skin, it is necessary for the applicator 1c to be pressed against the patient's skin to create a flattened area to receive the X-rays. The present invention allows the X-ray tube head 1 to be moved closer to the patient to press the applicator 1c onto the patient's skin. This means that the patient themselves do not need to be moved towards the X-ray tube head 1. The present invention therefore provides an easy and safe way of delivering X-ray radiotherapy to a patient.

Modifications and improvements may be made to the above without departing from the scope of the present invention.

For example, although the mounting apparatus 10 has been illustrated and described above as being used to support an X-ray tube head medical device, it should be appreciated that the mounting apparatus 10 may be used to support and adjust the position of other types of medical devices, or even other devices. With such an apparatus, the mounting apparatus for a device would comprise: a support member; a device engagement member, the device engagement member being moveably engageable with the support member; and an adjustment device, the adjustment device being configured to move the device engagement member relative to the support member. It will be appreciated that the medical device engagement member 14 would be a device engagement member.

For example, although the structure attachment portion 16 has been illustrated and described above as being attachable to a structure which is a base unit 4 of a medical device system, it should be appreciated that the structure may be a component of a building, such as a wall, or the like. Alternatively, the structure may be component of a vehicle, such as the wall of a building, such as a wall, or the like. In this arrangement, the support structure 10 may be attachable to a wall, vehicle wall, or the like.

1. A mounting apparatus for a medical device, comprising:
a housing support member;
a medical device engagement member, the medical device engagement member being moveably engageable with the housing support member; and
an adjustment device, the adjustment device being configured to move the medical device engagement member relative to the housing support member.

2-3. (canceled)

4. The mounting apparatus of claim 1, wherein the housing support member includes two oppositely-facing elongate apertures in the side wall thereof, the two apertures extending along the side wall on a plane orthogonal to a longitudinal axis of the mounting apparatus.

5. The mounting apparatus of claim 1, wherein the housing support member includes an adjustment device support, the adjustment device support being arranged to protrude from, and extend around, the outwardly facing surface of the side wall of the housing support member.

6. The mounting apparatus of claim 5, wherein the adjustment device support may be a channel portion, the channel portion being configured to receive at least a portion of the adjustment device therein.

7. The mounting apparatus of claim 1, wherein the housing support member incudes a channel portion, the channel portion being configured to receive at least a portion of the medical device engagement member therein and to allow relative linear translational movement between the housing support member and the medical device engagement member.

8. (canceled)

9. The mounting apparatus of claim 1, wherein the medical device engagement member includes two oppositely-facing elongate apertures in the side wall thereof, the two apertures extending along the side wall at an angle to a plane orthogonal to the longitudinal axis of the mounting apparatus.

10. The mounting apparatus of claim 9, wherein each aperture in the side wall of the medical device engagement member is arranged to extend in a horizontal and vertical direction from a lower, or bottom, portion of the medical device engagement member towards an upper, or top, portion of the medical device engagement member.

11. The mounting apparatus of claim 9 wherein each aperture in the side wall of the medical device engagement member includes an engaging member contact surface, the contact surface being configured to engage with an engaging member of the adjustment device.

12. The mounting apparatus of claim 9, wherein the angle of inclination of each of the two apertures of the side wall of the medical device engagement member makes with the plane may be between 6.5 degrees and 12.5 degrees from horizontal.

13. The mounting apparatus of claim 1, wherein the housing support member and the medical device engagement member are arranged such that relative linear translational movement between the two is permitted, and relative rotational movement about the longitudinal axis of the mounting apparatus between the two is prohibited.

14. The mounting apparatus of claim 7, wherein the medical device engagement member includes an attachment member, the attachment member being configured such that the medical device engagement member may be attachable to a medical device, or an attachment member of a medical device, wherein the engagement of the attachment member of the medical device engagement member in the channel portion of the housing support member prohibits relative rotational movement between the housing support member and the medical device engagement member about the longitudinal axis of the mounting apparatus.

15. (canceled)

16. The mounting apparatus of claim 1, wherein the adjustment device is configured to rotate about the longitudinal axis of the mounting apparatus between a first position and a second position, the first position corresponding to the first position of the medical device engagement member, and the second position of the body portion corresponding to the second position of the medical device engagement member.

17. (canceled)

18. The mounting apparatus of claim 1, wherein the housing support member includes two oppositely-facing elongate apertures in the side wall thereof, the two apertures extending along the side wall on a plane orthogonal to a longitudinal axis of the mounting apparatus,
wherein the medical device engagement member includes two oppositely-facing elongate apertures in the side wall thereof, the two apertures extending along the side wall at an angle to a plane orthogonal to the longitudinal axis of the mounting apparatus, and
wherein the adjustment device includes one or more medical device engagement member engaging members, the engaging members being arranged to engage the apertures in the side wall of the medical device engagement member via the apertures in the side wall of the housing support member.

19. The mounting apparatus of claim 18, wherein the engaging members of the adjustment device protrude from an inwardly facing surface of the side wall of the adjustment device, through the apertures in the side wall of the housing support member and into the apertures in the side wall of the medical device engagement member.

20. The mounting apparatus of claim 18 wherein the adjustment device is arranged such that the engaging members of the adjustment device protrude through the apertures in the side wall of the housing support member to engage with the apertures in the side wall of the medical device engagement member, such that rotation of the adjustment device relative to the housing support member from the first position to the second position causes the medical device engagement member to move between its first position and its second position.

21. The mounting apparatus of claim 18, wherein the apertures in the side walls of the housing support member and the medical device engagement member are angularly offset from one another, such that the engaging members of the adjustment device engage the contact surfaces of the apertures of the medical device engagement member and exert a force in the direction of the longitudinal axis of the mounting apparatus as the body portion of the adjustment device is rotated relative to the housing support member.

22. The mounting apparatus of claim 18, wherein the engaging members are pin members, the pin members being removably attached to the body portion of the adjustment device.

23. The mounting apparatus of claim 1, wherein the mounting apparatus is configured to be pivotably attachable to a support structure of a medical device system about at least two axes of rotation relative to the support structure.

24. (canceled)

25. A medical device system, comprising:
a base unit;
a support structure for supporting a medical device mounting apparatus, the support structure being attachable to the base unit; and
a mounting apparatus for a medical device, comprising:
a housing support member;
a medical device engagement member, the medical device engagement member being moveably engageable with the housing support member; and
an adjustment device, the adjustment device being configured to move the medical device engagement member relative to the housing support member.

26-27. (canceled)

28. A medical device system, comprising:
a base unit;
a support structure for supporting a medical device mounting apparatus, the support structure being attachable to the base unit; and
a mounting apparatus for a medical device, comprising:
a housing support member;
a medical device engagement member, the medical device engagement member being moveably engageable with the housing support member;
an adjustment device, the adjustment device being configured to move the medical device engagement member relative to the housing support member; and
a medical device mounted to the mounting apparatus.

29. (canceled)
